(19) United States
(12) Patent Application Publication
(10) Pub. No.: US 2026/0315686 A1
(43) Pub. Date: Oct. 15, 2026

(54) PROTECTIVE COVER/POUCH THAT SECURES A MEDICAL DEVICE

(71) Applicant: CATHCARE, LLC, Wainscott, NY (US)

(72) Inventor: Ellenka HUNTER, Wainscott, NY (US)

(21) Appl. No.: 19/482,503

(22) PCT Filed: May 10, 2024
(86) PCT No.: PCT/US2024/028804

§ 371 Date: Nov. 7, 2025

Related U.S. Application Data
(60) Provisional application No. 63/501,394, filed on May 10, 2023.

(51) Int. Cl. A61M 5/14

(52) U.S. Cl. CPC A61M 5/1415; A61M 2005/1416; A61M 2205/59; A61M 2209/088

(57) ABSTRACT

A protective cover/pouch assembly detachably securable to a wearer includes inner and outer surfaces with the inner surface disposed proximate the wearer and the outer surface disposed distal the wearer. The protective cover/pouch assembly includes a pocket/cavity accessible via an exterior opening. The pocket/cavity includes an interior slit/aperture in an inner oriented surface thereof for passing a medical device component therethrough. The protective cover/pouch can be formed using a stretchable material that conforms to the wearer. The protective cover/pouch assembly can include a viewing/window region including an aperture and an aperture cover that allows a medical device or component thereunder to be viewed and/or accessed and covered.

### CROSS-REFERENCE TO RELATED APPLICATIONS

This application claims the benefit, under Articles 4 and 8 of the Stockholm Act of the Paris Convention for the Protection of Industrial Property to U.S. Provisional Pat. App. No. 63/501,394, filed May 10, 2023, which application is incorporated herein by reference in its entirety.

### FIELD

The instant disclosure generally relates to a protective cover/pouch detachably securable to a wearer that secures and protects a medical device and/or components thereof, and more specifically to a protective cover/pouch detachably securable to a body part of a wearer, which safely contains the medical device and/or components therein, maintains cleanliness of the medical device and/or components therein, reduces the likelihood of damage to the medical device and/or components therein, and/or reduces the incidence of injury to the wearer, among other things.

### BACKGROUND

Intravenous (IV) therapy generally requires the administration of medications or fluids via a catheter inserted into a patient's vein. Although most IV therapy is short term (from minutes to a few days), there are many instances in which an extended duration of IV therapy is required. For example, in the case of long-term treatment with antibiotics or cancer chemotherapy, IV therapy can be performed over several days to several weeks. For such extended duration, specialized long-term catheters are typically often used. Examples of such specialized long-term catheters include peripherally-inserted central line catheters (PICC lines) or extended length peripheral catheters (midlines). In such cases, patients are often sent home with the long-term catheters implanted in site for continued IV therapy at home.

As may be imagined, there are some problems associated with extended IV therapy or home care. That is, the IV catheter requires routine cleaning and/or dressing changes. Moreover, the port on the external end of the IV catheter must be protected from dirt, oil, debris, microbes, and other types of potential contamination. Additionally, IV catheters have tubing parts and lengths of tubing that must be secured to prevent accidental dislodgement and/or must be stored when not in use. While medical tape, elastic wrapping, sleeves and/or nettings are be cumbersome. For example, the use of tapes can often leave adhesive residues that may be irritating to the skin or hair. Furthermore, the use of tapes, wraps, or sleeves require that such materials be removed to access the IV catheter or lines, if needed, and then re-applied once the IV therapy is complete. The removal and re-application of tapes, wrappings, and sleeves can result in significant irritation and possible dislodgement, causing additional pokes to the patient. Moreover, when the IV catheter or associated components are readily visible, such can serve as a reminder the patient, or others, of the patient's medical condition, which can be uncomfortable to the patient and/or detrimental to the patient's emotional well-being.

What is needed then, is a protective cover/pouch that is capable of comfortably securing an IV catheter and its associated components from damage, dislodgement, and/or contamination, which protective cover/pouch allows the patient and/or medical personnel to readily access the IV catheter and/or its associated components without necessarily requiring removal thereof. What is also needed is a protective cover/pouch capable of storing a medical device and components therefor when not in use, which protective cover/pouch is comfortable to wear, cosmetically pleasing, and can lend a sense of normalcy to the wearer, especially, for example, in the case of young patients or geriatrics who are forgetful and or physically weak.

### SUMMARY

At the outset it should be understood that while the following disclosure, figures, and/or claims, etc. describe subject matter including one or more aspects described as either alone or in combination with one or more other aspects, the subject matter of the instant disclosure is not intended to be so limited. That is, the instant disclosure, figures, and claims are intended to encompass the various aspects described herein, either alone or in one or more combinations with one another. For example, while the instant disclosure may describe and illustrate a first aspect, a second aspect, and a third aspect in a manner such that the first aspect is only specifically described and illustrated relative to the second aspect, or the second aspect is only described and illustrated relative to the third aspect, the instant disclosure and illustrations are not intended to be so limiting and may encompass the first aspect alone, the second aspect alone, the third aspect alone, or one or more combinations of the first, second, and/or third aspects, e.g., the first aspect and the second aspect, the first aspect and the third aspect, the second and third aspect, or the first, second and third aspects.

for a medical device that is detachably securable to a wearer. The protective cover/pouch assembly has a first end and a second end and includes a cover/guard portion having an inner surface and an outer surface, the inner surface is configured to be disposed proximate the wearer and the outer surface configured to be disposed distal the wearer. The protective cover/pouch assembly further includes a pouch assembly portion disposed between the first end and the second end, the pouch assembly portion includes a cavity disposed at one or more of a position that is between the inner surface and the outer surface of the cover/guard portion, on the inner surface of the cover/guard portion, or on the outer surface of the cover/guard portion. The cavity is accessible via an exterior opening in an outer oriented surface of the pouch assembly portion, the exterior opening is openable and closable via one or more of an elastically deformable member, an overlapping flap assembly, a zipper, a hook and loop-type fastener assembly, one or more snaps or buttons, a cinching member, or a re-sealable sliding channel sealing assembly. In addition to the above, the cavity includes at least one interior slit/aperture disposed in an inner oriented surface of the pouch assembly portion such that a medical device and/or a medical device component can be passed therethrough. Finally, one or more of the cover/guard portion and the pouch assembly portion comprises a stretchable and elastically deformable material that conforms with a portion of the wearer's body.

In some aspects, the inner surface comprises the stretchable and elastically deformable material and is formed in the manner of a sleeve member configured to receive the portion of the wearer's body, e.g., the sleeve can receive a wearer's arm, therethrough. In some aspects, the protective cover/pouch assembly includes at least one strap member configured to wrap and secure about the portion of the wearer's body, e.g., an elastic strap and fastener, e.g. hook and loop, that secures around a wearer's arm.

In some aspects, at least one of the cover/guard portion and/or the pouch assembly portion comprises one or more of a breathable fabric, a moisture wicking fabric, an anti-microbial fabric, and a cushioning material. In some aspects, at least one of the cover/guard portion and/or the pouch assembly portion comprises at least one of a mesh or a plurality of vent holes. at least one of the cover/guard portion and/or the pouch assembly portion comprise a foam, e.g., stretchable neoprene foam.

In some aspects, the cavity of the pocket assembly portion is formed from at least least one side wall comprises at least one of a breathable fabric, a moisture wicking fabric, an anti-microbial fabric, and a cushioning fabric. In some aspects, the at least one side wall comprises at least one of a mesh or a plurality of vent holes.

In some aspects, the protective cover/pouch assembly additionally includes a viewing window/region disposed between the first end and the second end. In some aspects, the viewing window/region is disposed toward the first end and the pouch assembly region is disposed toward the second end. In some aspects, the viewing window/region includes an aperture formed in the cover/guard portion. In some aspects, the aperture is accessible via an openable and closable aperture cover disposed over the aperture.

In some aspects, the aperture and the aperture cover are configured to be disposed over a catheter hub portion of a mounted catheter assembly, and the pouch assembly portion is configured to be disposed over a catheter port portion of the mounted catheter assembly. In some aspects, the catheter port portion is configured to be passed through at least one slit/aperture disposed in the inner oriented surface of the pouch assembly portion and received within the cavity. In some aspects, the aperture cover includes a flap portion and a hinge portion that is secured to the cover/guard portion.

In some aspects, the hinge portion is disposed in a direction that is transverse/perpendicular a longitudinal axis of the protective cover/pouch assembly or in a direction that is parallel to the longitudinal axis. In some aspects, the aperture cover includes at least one male/female fastener assembly, and a portion of the cover/guard portion bordering the aperture includes a corresponding at least one female/male fastener assembly that complementarily fastens to the male/female fastener assembly of the aperture cover. In some aspects, the at least one male/female fastener and corresponding at least one female/male fastener assembly comprises at least one or more of hook and loop, a zipper, a snap, a button, a magnetic connection, a resealable sliding channel sealing assembly, e.g., a ZipLoc® type assembly, etc. In some aspects, the aperture cover includes at least one pull tab allowing the aperture cover to be gripped. In some aspects, the aperture cover includes indicia indicating the potential that a medical device or a medical component is disposed below the aperture cover.

In some aspects, at least one of the cover/guard, the pocket, or the aperture cover comprises one or more of a skin-tone hue, a hue corresponding to one or more colors, a pattern, a logo, corporate logo, or a superhero, etc.

In some aspects, the protective cover/pouch assembly further includes an elastic band portion disposed proximate the first end, which conforms to a portion of the wearer's body, e.g., to provide a better fit. In some aspects, the protective cover/pouch assembly includes a non-slip member, such as silicone, disposed on an inner surface of the cover/guard so as to prevent movement of the protective cover/pouch, e.g. rotational movement or sliding movement.

These and other aspects, features, and advantages of the present disclosure will be readily appreciable from the following description in view the accompanying drawings and claims.

### BRIEF DESCRIPTION OF THE DRAWINGS

The nature and mode of operation of an invention in accordance with the instant disclosure will now be more fully described in the following detailed description of the invention taken with the accompanying drawing figures, in which:

FIG. 1 is a view of a current method of securing an IV catheter and associated components to a patient;

FIG. 2A is a perspective view of a protective cover/pouch assembly according to the instant disclosure detachably secured to a body part of a patient, e.g., an arm of a patient, which shows a cover of a viewing aperture/window in a closed position;

FIG. 2B is a perspective view of a protective cover/pouch assembly according to the instant disclosure detachably secured to a body part of a patient, e.g., an arm of a patient, which shows an aperture cover of a window/aperture in an open position;

FIG. 2C is a perspective view of a second end of a protective cover/pouch assembly according to the instant disclosure detachably secured to a body part of a patient, e.g., an arm of a patient, which shows a pocket in an open position and showing slit/aperture therein;

FIG. 2D is a perspective view of a first end of a protective cover/pouch assembly according to the instant disclosure, which shows an elastic band and anti-slip rubber portion;

FIG. 3A is front and rear views of a protective cover/pouch assembly according to the instant disclosure detachably secured to a body part of a patient, e.g., an arm of a patient, which shows an aperture cover of a window/aperture having a hinge that is disposed transverse a longitudinal axis thereof with the aperture cover in an open position; instant disclosure detachably secured to a body part of a patient, e.g., an arm of a patient, which shows an aperture cover of a window/aperture having a hinge that is disposed parallel with a longitudinal axis thereof, with the aperture cover in open and closed positions, and with the pocket in open and closed positions;

FIG. 3C is front and rear views of a protective cover/pouch assembly according to the instant disclosure detachably secured to a body part of a patient, e.g., an arm of a patient, which shows an aperture cover of a window/aperture having a hinge that is disposed transverse a longitudinal axis thereof, with the cover in open and closed positions, and with the pocket in open and closed positions;

FIG. 3D is front and rear views of a protective cover/pouch assembly according to the instant disclosure detachably secured to a body part of a patient, e.g., an arm of a patient, which shows an aperture cover of a window/aperture having a hinge that is disposed parallel with a longitudinal axis thereof, with the cover in open and closed positions, and with the pocket in open and closed positions;

FIG. 4A is front and rear views of a protective cover/pouch assembly according to the instant disclosure detachably secured to a body part of a patient, e.g., an arm of a patient, which shows an internally disposed pocket in open and closed positions;

FIG. 4B is front and rear views of a protective cover/pouch assembly according to the instant disclosure detachably secured to a body part of a patient, e.g., an arm of a patient, which shows an internally disposed pocket in open and closed positions;

FIG. 5A-5B are front and rear views of protective cover/pouch assemblies according to the instant disclosure detachably secured to a body part of a patient, e.g., an arm of a patient, which shows various types of pockets.

### DETAILED DESCRIPTION

At the outset, it should be appreciated that like drawing numbers on different drawing views identify identical, or functionally similar, structural elements. It is to be understood that the claims are not limited to the specifically disclosed and illustrated aspects.

Furthermore, it is understood that this disclosure is not limited to the particular methodologies, materials and modifications described and as such may, of course, vary. It is aspects only and is not intended to limit the scope of the disclosure or claims.

Unless defined otherwise, all technical and scientific terms used herein have the same meaning as commonly understood to one of ordinary skill in the art to which this disclosure belongs. Although any methods, devices or materials similar or equivalent to those described herein can be used in the practice the example aspects.

It should be understood that use of “or” in the present application is with respect to a “non-exclusive” arrangement, unless stated otherwise. For example, when saying that “item x is A or B,” it is understood that this can mean one of the following: (1) item x is only one or the other of A and B; (2) item x is both A and B. Alternately stated, the word “or” is not used to define an “exclusive or” arrangement. For example, an “exclusive or” arrangement for the statement “item x is A or B” would require that x can be only one of A and B. Moreover, as used herein, the phrases “comprises at least one of” and “comprising at least one of” in combination with a system or element is intended to mean that the system or element includes one or more of the elements listed after the phrase. For example, a device comprising at least one of: a first element; a second element; and, a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or, a device comprising a second element and a third element. A similar interpretation is intended when the phrase “used in at least one of:” is used herein. Furthermore, as used herein, “and/or” is intended to mean a grammatical conjunction used to indicate that one or more of the elements or conditions recited may be included or occur. For example, a device comprising a first element, a second element and/or a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or, a device comprising a second element and a third element.

synonymous with terms such as “nearly,” “very nearly,” “about,” “approximately,” “around,” “bordering on,” “close to,” “essentially,” “in the neighborhood of,” “in the vicinity of,” etc., and such terms may be used interchangeably as appearing in the specification and claims. It should be appreciated that the term “proximate” is synonymous with terms such as “nearby,” “close,” “adjacent,” “neighboring,” “immediate,” “adjoining,” etc., and such terms may be used interchangeably as appearing in the specification and claims. The term “approximately” is intended to mean values within ten percent of the specified value.

While the following terms are not to be specifically limited, “non-rotatably connected” elements, is intended to refer to that the elements are connected so that whenever one of the elements rotate, all the elements rotate; and relative rotation between the elements is not possible. Radial and/or axial movement of non-rotatably connected elements with respect to each other is possible, but not required. Additionally, “plastic deformation” is intended to refer to, for example, instances wherein when a sufficient load is applied to a material to form it into a desired shape and such force released, the material is formed into the desired shape and the desired shape maintained until a further sufficient force may be applied so as to reform the material. In other words, a material subject to plastic deformation may be subject to a sufficient force so to bend or form such material to maintain a first shape, and then may be subject to a subsequent sufficient force to bend or reform such material to return it so as to maintain its original shape, or to maintain a further shape. “Elastic deformation” is intended to refer to instances wherein when a sufficient load is applied to a material such material undergoes a temporary shape change while subject to such force but is restored to its original shape upon release of such force. “Resilient” can refer to the ability of a material to return to its original form and/or have force absorbing/rebounding qualities. Hence, a material can be resilient in the sense that upon application of a force it rebounds entirely to return to its original form, e.g., some rubbers, non-resilient in that it has no rebound and does not return to its original form, e.g., some hard plastics and metals, or semi-resilient in that it only partially rebounds and only partially returns to its original form, e.g., some plastics, rubbers and foams, etc.

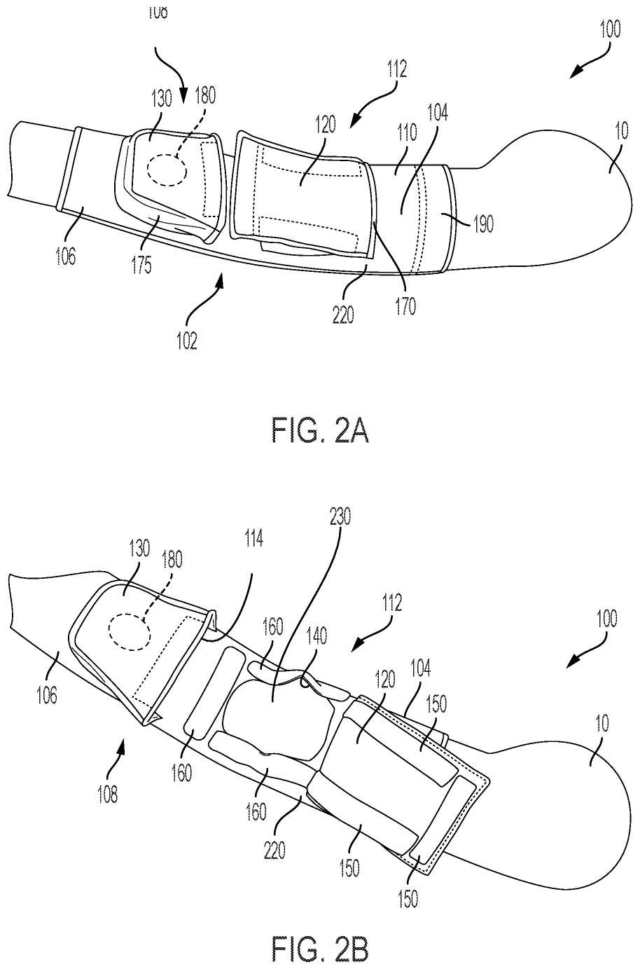

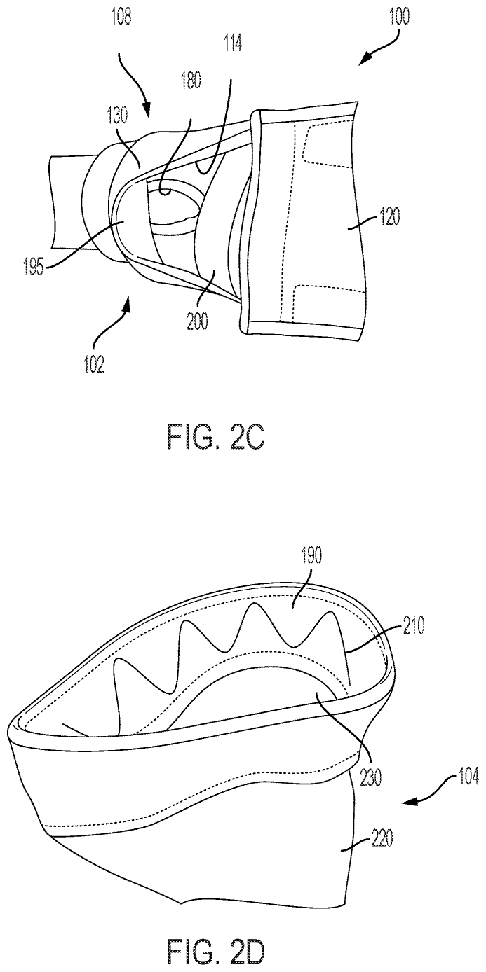

Also, the descriptors “outer”, “inner”, “upper”, “lower”, “top”, “bottom”, “right”, “left”, and their derivatives in the description herebelow refer to the perspective of one facing the inventive subject matter as shown in, for example, FIGS. 2A and 2B. Additionally, it should be inventive subject matter and, unless otherwise specified, is not intended to specifically define one or more sides, or surfaces, as such.

It should be appreciated that while the instant disclosure primarily describes a protective cover/pouch assembly used in association with an IV catheter or its associated components and an arm of a wearer, e.g., a catheter port and/or tubing, the protective cover/pouch assembly is not intended to be limited to IV catheters or their associated components and/or an arm of a wearer, for example, but rather, can be configured to receive other devices and/or applied to other parts of a wearer, e.g., arm, elbow, upper arm, chest, stomach, leg, hip, etc., and/or be configured so as to accommodate various types of potential wearers, e.g., children, adults, tall, short, large, small, animals, etc.

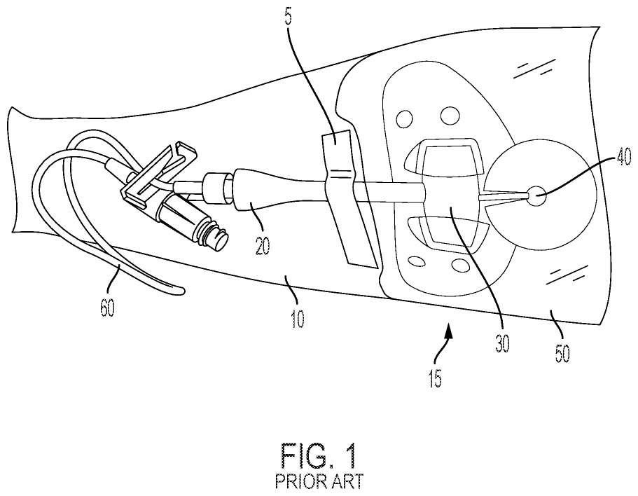

Adverting now to the figures, in which FIG. 1 illustrates a current method by which a catheter and its associated components can be secured to an arm of patient, and FIGS. 2A-5B, which illustrate various aspects of a protective cover/pouch assembly in accordance with the instant disclosure and the inventive subject matter.

As shown in FIG. 1, patient/patient arm 10 is generally shown as having catheter assembly 15 inserted into insertion point 40 of an arm of the patient. Catheter assembly 15 is generally shown as including catheter port 20, catheter hub 30, and catheter tube 60. Catheter hub 30 and catheter insertion point 40 are typically covered with a clear adhesive bandage 50, which allows the wearer to readily view catheter hub 30 and insertion point 40 without the need to remove adhesive bandage 50. Catheter port 20 extends from catheter hub 30 via a tube member, which tube member is typically secured to patient 10 by means of a length of adhesive tape 55. Currently, when catheter assembly 15 and its various associated components are not in use, they are typically covered and secured to patient 10 by means of wraps or bandages that serve to prevent damage to the catheter assembly, dislodgement of the catheter assembly from the patient, and/or serve to maintain cleanliness of the catheter assembly and insertion point, for example. The use of wraps and bandages can be burdensome in that they can be difficult for the patient to apply or remove by his/herself, can be difficult for others to apply, and/or do not provide ready access to the catheter assembly and/or the catheter insertion point.

Referring now to FIGS. 2A-5B, as shown in FIGS. 2A and 2B, protective cover/pouch 100 for a medical device such as catheter assembly 15 is shown as generally formed Protective cover/pouch 100 generally includes cover/guard portion 102 extending from first end 104 to second end 106, which is in the manner of a tubular sleeve 110 that forms a tubular cavity. Protective cover/pouch 100 is shown as additionally including pouch assembly portion 108 and window/viewing portion 112. As may be appreciated, in the case of a protective cover/pouch for use with a patient's arm whose bicep portion of their arm may be larger than a forearm portion of their arm, tubular sleeve 110 can be tapered and/or configured such that first end 104 has a diameter that is greater than second end 104, which helps in allowing the tubular sleeve 110 to be easily slid on by a user with one hand, while snugly securing to the wearer's arm. As may also be appreciated, protective cover/pouch 100 and its associated components can be made in various lengths, widths, sizes, and orientations, so as to secure to various portions of a patient's body, e.g., arm, elbow, upper arm, chest, stomach, leg, hip, etc., and/or to accommodate various types of potential wearers, e.g., children, adults, tall, short, large, small, animals, etc.

In some aspects, cover/guard portion 102 can formed from one or more stretchable and elastically deformable materials/fabrics that generally apply a compressive force to conform to the shape of the body part of a patient to which it is secured. For example, in the case of patient arm 10, the cover/guard portion 102 can apply a compressive force to patient arm 10 such that it conforms to the shape of the patient arm and additionally secure a medical device or medical device components disposed thereunder, e.g., catheter assembly 15.

In some aspects, cover/guard portion 102 can be formed from one or more fabrics and materials that can have skin-safe, breathable, cushioning, anti-microbial, waterproof, etc., qualities. Examples of suitable materials include stretchable neoprene foams, spandex, meshes, netted-type fabrics, etc. Additionally, cover/guard portion 102 can comprise a multilayer structure formed from a plurality of materials having one or more properties. For example, an inner layer 230 intended to be disposed against a patient's skin can be formed from a stretchable, skin-safe, anti-microbial breathable mesh fabric, a mid-layer (not shown) can be formed from a stretchable foam, and an outer layer 220 can be formed from a stretchable fabric that may have waterproof or water-resistant properties. As may also be appreciated, the multilayer structure is not limited to the specific examples discussed herein.

strap or band members 190 for additionally supporting the protective cover/pouch 100 to the wearer. In some aspects, the one or more strap or band members 190 can be in the manner of an elastic band secured to an inner or outer surface of the cover/guard portion. In the case of a multilayer structure, one or more strap or and members can be disposed between one or more layers thereof. Additionally, one or more strap or band members can comprise an adjustable strap/buckle assembly and/or an appropriate fastener, e.g., hook and loop, snaps, buttons, etc. As may be appreciated, while FIGS. 2A and 2D illustrate a single strap or band member 190 disposed toward first end 104, the one or more strap members can be disposed at various positions along the length of cover/guard portion 102. For example, a first strap or band member 190 can be disposed proximate first end 104, a second strap or band member 190 can be disposed proximate second end 106, and a third strap or band member 190 can be disposed at a position between pouch assembly portion 108 and window/viewing portion 112. By way of further example, in the case of a patient's arm, the third strap or band member 190 described above can be disposed at a position proximate an elbow joint of a patient. Finally, as shown in FIG. 2D, cover/guard portion 102 can include one or more non-slip members 210 disposed on the inner surface of cover/guard portion 102. Non-slip members 210 can be formed from rubber or silicone and are generally provided for gripping clothing of a wearer so as to prevent movement of the cover/guard portion relative to the patient, e.g., preventing linear or rotational movement of the cover/guard portion along its longitudinal axis.

As shown in FIGS. 2A-2C, pouch assembly portion 108 is generally provided for receiving and storing a medical device or a medical device component therein, e.g., catheter tubing, when not in use. Pouch assembly portion 108 includes pocket 130 having a cavity formed from one or more pocket walls or side walls 175. The one or more pocket walls or sidewalls 175 are secured to and extend from an outer surface of cover/guard portion 102 so as to allow pocket 130 to expand in size in order to accommodate various medical devices, components, and/or lengths of tubing that may be stored therein. As shown more clearly in FIG. 2A, pocket 130 includes an internal slit/aperture 180 that extends from the pocket cavity and into the tubular cavity of the cover/guard portion, which allows a medical device, components therefor, or tubing to pass from the tubular cavity of cover/guard portion 102 and into the pocket cavity. As shown in FIGS. 2A-2C, the cavity of pocket 130 is additionally accessible via closed position via one or more of an elastically deformable member, e.g., an elastic band that applies a compressive force, an overlapping flap assembly, one or more various types of mating fastener, such as hook and loop 150/160/195/200, zipper assemblies 520, one or more snaps or buttons 610, a cinching member, a resealable sliding channel sealing assembly, e.g., a ZipLoc® type assembly, magnets, etc. Additionally, it should be appreciated that while exterior opening 114 is shown as being disposed in a direction that is generally perpendicular to the longitudinal axis of the protective cover/pouch, it may be otherwise oriented, for example, exterior opening 114 can be oriented parallel, or diagonal, with the longitudinal axis.

In some aspects, pouch assembly portion 108/pocket 130 can be formed from one or more stretchable and elastically deformable materials/fabrics that generally apply a compressive force to conform to the shape of a patient's body. In some aspects, pouch assembly portion 108/pocket 130 can be formed from one or more fabrics and materials that can have skin-safe, breathable, cushioning, anti-microbial, and/or waterproof, etc., qualities. Examples of suitable materials include stretchable neoprene foams, spandex, meshes, netted-type fabrics, etc. Additionally, pouch assembly portion 108/pocket 130 can comprise a multilayer structure formed of a plurality of materials having one or more properties. For example, an inner surface of pocket 130 can be formed of breathable fabric having anti-microbial properties, a second mid-layer can be formed from a stretchable foam, and a third outer layer can be formed from a stretchable fabric that may have waterproof or water-resistant properties. Additionally, one or more walls of pocket 130 can be formed from one or more materials. For example, one or more side walls 175 of pocket 130 can be formed from a first material, e.g., mesh, and an outer exterior wall formed from a second material, e.g., a stretchable neoprene foam. As may be appreciated, the multilayer structure/pocket wall structures are not limited to the specific discussed examples.

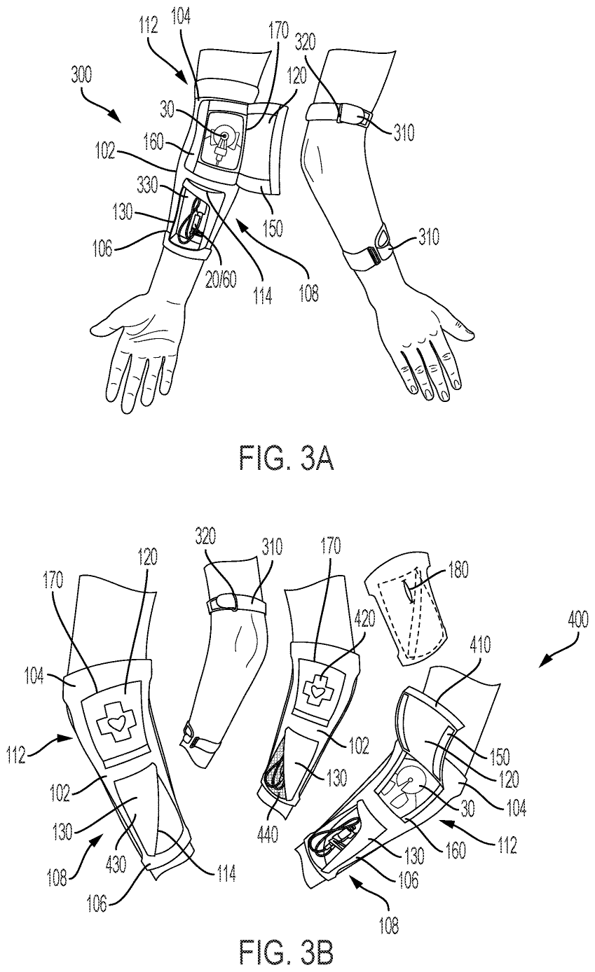

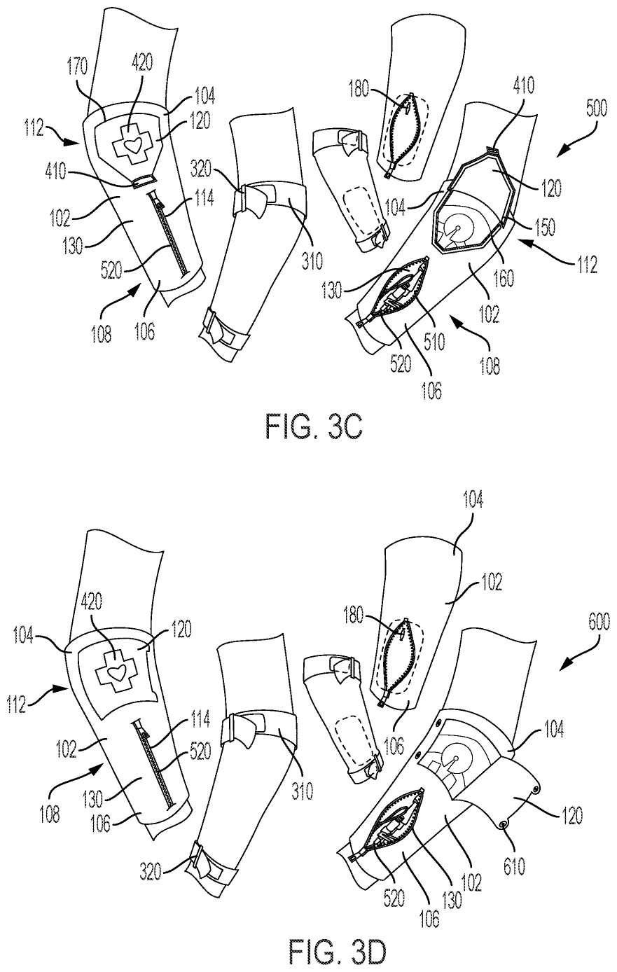

As shown in FIGS. 2A-2B, window/viewing portion 112 is generally provided for protecting and allowing ready access/viewing of a medical device or medical device component disposed below aperture cover 120. In the case of, for example, a patient's arm 10 having a catheter assembly inserted therein, window/viewing portion 112 and aperture cover 120 serve to protect catheter hub 30 and catheter insertion point 40, yet also allow catheter hub 30 and catheter insertion point 40 to be readily viewed and accessed. In accordance therewith, window/aperture 140. Aperture cover 120 can include one or more male/female fasteners 150 disposed on an inner surface thereof, which are configured for complementary mating with female/male fasteners 160 of cover/guard portion 102 bordering window/aperture 140. Examples of fasteners 150/160 include one or more of hook and loop, zipper assemblies, snaps, buttons, magnets, resealable sliding channel sealing assemblies, e.g., ZipLoc® type assemblies, etc. Aperture cover 120 can be hingedly secured to cover/guard portion 102 via hinge 170. Hinge 170 can be oriented parallel to the longitudinal axis of the protective cover/pouch 100 and disposed on either a leftward or rightward sides of window/aperture 160 such that aperture cover 120 can be opened and closed in leftward or rightward manners as viewed from the front of a wearer, see additionally, e.g., FIGS. 3A and 3D. Alternatively, hinge 170 can be oriented perpendicular to the longitudinal axis of the protective cover/pouch 100 and above or below window/aperture 160 such that aperture cover 120 can be opened in an upward or downward manner as viewed from the front of a wearer, see additionally, e.g., FIGS. 3B and 3C.

In some aspects, window/viewing portion 112 and aperture cover 120 can be formed from one or more stretchable and elastically deformable materials/fabrics that generally apply a compressive force so as to conform to the shape of a patient's body. In some aspects, window/viewing portion 112 and aperture cover 120 can be formed from one or more fabrics and materials that can have skin-safe, breathable, moisture-wicking, cushioning, anti-microbial, and/or waterproof, etc., qualities. Examples of suitable materials include stretchable neoprene foams, spandex, meshes, netted-type fabrics, etc. Additionally, window/viewing portion 112 and aperture cover 120 can comprise a multilayer structure formed from a plurality of materials having one or more properties. For example, an inner surface of aperture cover 120 can be formed of breathable fabric having anti-microbial properties, a second mid-layer can be formed from a stretchable foam, and a third outer layer can be formed from a stretchable fabric that may have waterproof or water-resistant properties. As may be appreciated, the multilayer structure/aperture cover structures are not limited to the specific discussed examples. Additionally, it should be further appreciated that while aperture cover 120 of FIGS. 2A-2C is shown as having a generally square or rectangular shape, aperture cover 120 can be other shapes, e.g., round or oval, or have rounded edges so as to prevent aperture cover 120 from getting caught on objects. Finally, owing to the fact that some medical treatments often require that the portion 102, pouch assembly portion 108, or window/viewing portion 112 can include mechanisms for providing heat or cooling to such regions. Such mechanisms can include, for example, electrically powered resistive elements, hot or cold-water/ice packs, or chemical heating and cooling elements that can be inserted and removed as needed, e.g., hot water/cold/ice packs or chemical heating/cooling elements can be placed behind aperture cover 120 for a time and then removed as necessary.

As may be appreciated from the figures, in the case of protective cover/pouch 100 used in association with a patient arm 10 and a catheter assembly 15, pouch assembly portion 108 is generally configured to be disposed proximate second end 106 and window/viewing portion 112 is generally configured to be disposed proximate first end such that aperture/window 140 and aperture cover 120 are positioned over catheter hub 30 and catheter insertion point 40. From window/aperture 140, catheter port 20 and/or its associated catheter tubing can be passed below s cover/guard portion 102 between window/aperture 140 and pocket 130, and then passed through slit/aperture 180 of pocket 130 for storage within pocket 130. As a result, when a catheter and its associated components are not in use, they can be comfortably secured within pocket 130 and under cover/guard portion 102, and aperture cover 120 thereby preventing damage, dislodgement, and/or contamination of the catheter and its components. Additionally, protective cover/pouch 100 allows the patient and/or medical personnel to readily access the catheter and its associated components when needed, without requiring removal of the protective cover/pouch 100. Moreover, protective cover/pouch 100 serves to keep the catheter and its components hidden from view, which can lend a sense of normalcy to the wearer, especially, for example, in the case of young patients. It should be understood that the exterior surface of protective cover/pouch 100 and one or more of the cover/guard 102, pocket 130, or the aperture cover 120 can comprise one or more of a skin-tone hue, a hue corresponding to one or more colors, a pattern, a design, or artwork, which can lend a sense of normalcy to the wearer. Also, aperture cover 120 can include indicia indicating the potential that a medical device is disposed thereunder. Finally, while protective cover/pouch 100 is described and illustrated as being in the form of a continuous tubular sleeve, and while it is not shown in the figures, it can be adapted to include a zipper or other fastener, for example, that extends along its longitudinal length or along separated from one another.

Referring now to FIGS. 3A-3D, which illustrate additional aspects of a protective cover/pouch in accordance with the instant disclosure and the inventive subject matter. As may be appreciated, because protective cover/pouch 300/400/500/600 of FIGS. 3A-3D include and/or can include several features described relative to protective cover/pouch 100 in FIGS. 2A-2D, such features are not rediscussed herebelow, but it is to be understood that they can be present. As shown in FIGS. 3A-3D, protective cover/pouch 300/400/500/600 is generally shown as including first end 104, second end 106, cover guard portion 102, pouch assembly portion 108, and window/viewing portion 112.

However, unlike protective cover/pouch 100, which is described as including a tubular sleeve, protective cover/pouch 300/400/500/600/800 is configured to be secured via one or more adjustable straps 310 and buckles 320 (and/or fasteners therefor, e.g., hook and loop) for securing the device to an arm of a user. While FIGS. 3A and 3B illustrate a pair of adjustable straps 310 and buckles 320 wherein a first one thereof is disposed proximate first end 104, and a second thereof disposed proximate second end 106, additional straps and buckles can be provided. Adjustable straps 310 can be formed from various materials including stretchable and non-stretchable fabrics. Additionally, FIGS. 3A and 3D illustrate that hinge 170 of aperture cover 120 can be disposed parallel to the longitudinal axis of the protective cover/pouch 300/600, or as shown in FIGS. 3B and 3C can be disposed perpendicular to the longitudinal axis of the protective cover/pouch 400/500.

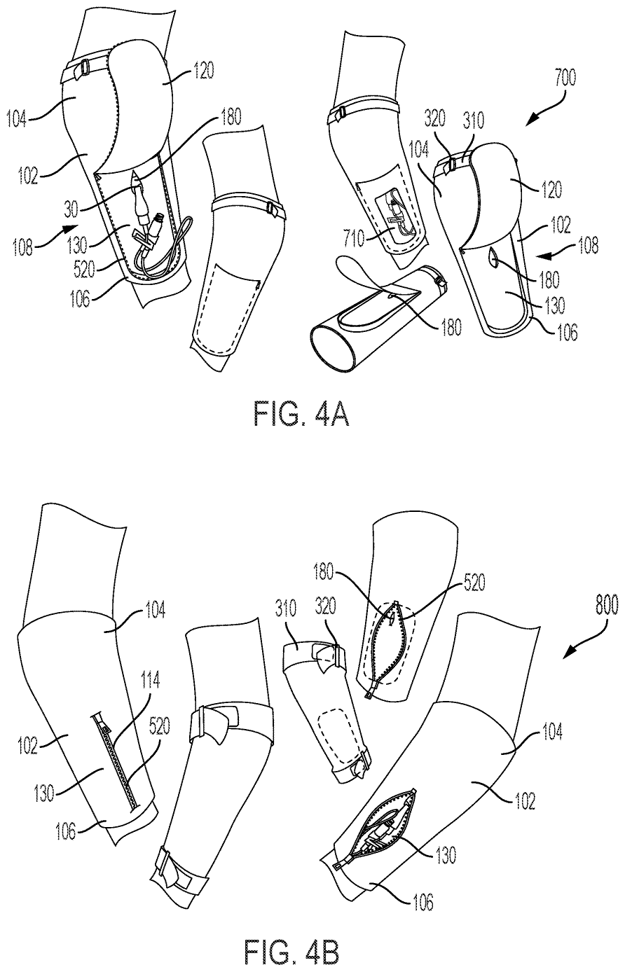

Furthermore, with regard to pocket 130, FIG. 3A illustrates that pocket 130 can include opening 114 openable and closable via an elastically deformable member, e.g., an elastic band or the force applied by the stretchable material from which it is made. Pocket 130 of FIG. 3A is shown as including cutaway portion 330 for purposes of illustrating that one or more catheter components 20/60 can be stored therein. FIG. 3B illustrates that pocket 130 can include opening 114 in the manner of overlapping flaps 430. FIG. 3B further illustrates that pocket 130 can comprise a plurality of materials, such as netting 440 for allowing air flow. FIGS. 3B and 3C also illustrate that aperture cover 120 can include pull tab portion 410 for more easily gripping aperture cover 120, as well a medical indicia 420 indicating that a medical device may be present under aperture cover 120. FIG. 3B also shows, in exploded/hidden view that component therethrough. FIGS. 3C, 3D, 4A and 4B additionally illustrate that pocket 130 can be in the form of an internal pocket accessible via a zipper assembly 520. FIG. 3D illustrates that aperture cover can include snap or button-type fasteners 610.

Referring now to FIGS. 4A-4B, which illustrate additional aspects of a protective cover/pouch in accordance with the instant disclosure and the inventive subject matter. As may be appreciated, because protective cover/pouch 700/800 of FIGS. 4A-4B include and/or can include several features described relative to protective cover/pouch 100/300/400/500/600 in FIGS. 2A-3D, such features are not rediscussed herebelow, but it is to be understood that they can be present. As shown in FIGS. 4A-4B, protective cover/pouch 700/800 is shown generally as including first end 104, second end 106, cover/guard portion 102, and pouch assembly portion 108, but while cover guard portion 102 is configured to cover catheter hub 30 and insertion point 40, it is shown as not including a window/viewing portion 112, aperture 140, or aperture cover 120.

As shown in FIG. 4A, protective cover/pouch 700 is in the manner of a tubular sleeve and includes pocket 130, which is externally accessible via zipper assembly 520 that extends along three sides of pocket 130. While hinge 170 formed by zipper assembly 520 is shown as being disposed perpendicularly relative to the longitudinal axis of the protective cover/pouch 700, it can be oriented parallel to the longitudinal axis. As shown in FIG. 4A, pocket 130 can include transparent window 710.

As shown in FIG. 4B, protective cover/pouch 800 includes pocket 130, which is externally accessible via zipper assembly 520 that extends in a direction parallel with longitudinal axis of the protective cover/pouch 800. While zipper assembly 520 is shown as being disposed parallel with the longitudinal axis of the protective cover/pouch 800, it can be oriented perpendicular to the longitudinal axis.

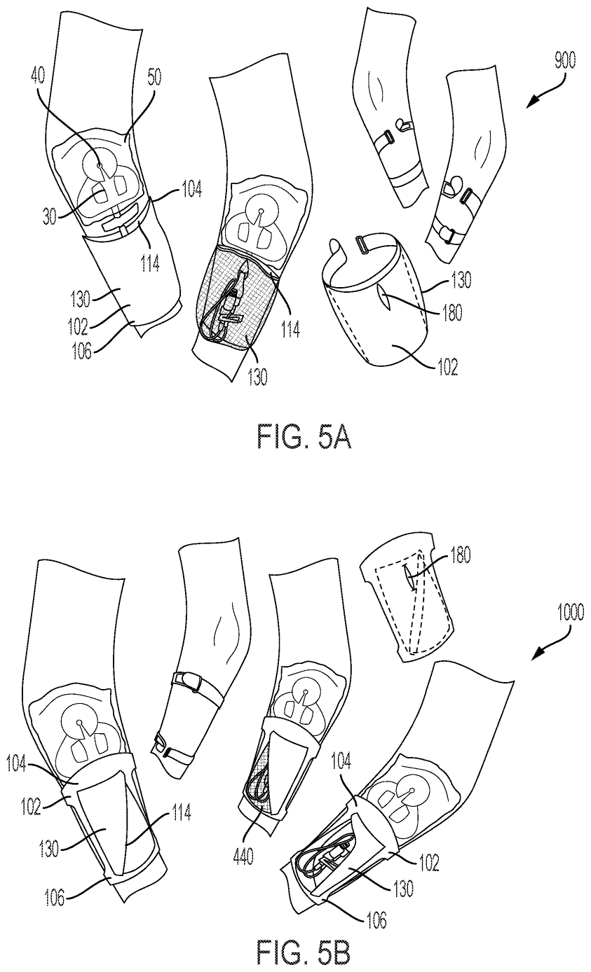

Referring now to FIGS. 5A and 5B, which illustrate so-called “shortened” protective covers/pouches 900/1000. As may be appreciated from FIGS. 5A and 5B, “shortened” protective covers/pouches 900/1000 are shown as generally including first end 104, second end 106, cover guard portion 102 and pouch assembly portion 108. However, cover guard portion 102 is not configured to cover a catheter hub 30 or insertion point 40. As shown in FIG. 5A, pocket 130 can include opening 114 openable and closable via an elastically which it is made. FIG. 5B illustrates that pocket 130 can include opening 114 in the manner of overlapping flaps 430. FIGS. 5A and 5B further illustrate that pocket 130 can comprise a plurality of materials, such as netting 440 for allowing air flow.

While the various aspects of a protective cover/pouch and its associated components have been generally described in association with an arm of a human body, it is to be understood that protective cover/pouch and its associated components can be made in various lengths, widths, sizes, and orientations, so as to secure to various portions of a patient's body, e.g., hand, arm, elbow, upper arm, chest, stomach, leg, hip, etc., and/or be configured to accommodate various types of potential wearers, e.g., children, adults, tall, short, large, small, animals, etc. For example, in the case of a protective cover/pouch for a chest region of a wearer, while not shown in the figures, cover/guard portion 102 can be in the form of a stretchable elastic fabric having one or more layers as previously described and configured in the form of a flat panel having one or more of pouch assembly portion 108/pocket 130, window viewing portion 112/window/aperture 140, and aperture cover 120, which can be wrapped about a patient's mid-section. Appropriate fasteners, such as hook and loop, zipper assemblies, snaps, buttons, magnets, a resealable sliding channel sealing assemblies, e.g., ZipLoc® type assemblies, etc. can be disposed proximate the first end 104 and second end 106 of cover/guard portion 102 and fastened to one another, either at the chest region or the back region of the wearer.

Additionally, in the case of protective covers/pouches for use in association with, for example, catheters inserted into a hand or foot of a patient, which may require a small footprint, such protective covers/pouches and their associated components can be made in various lengths, widths, sizes, dimensions, and orientations to accommodate such smaller footprints. Along this line, the protective covers/pouches described herein can include adhesive portions for adhering to various body parts, e.g., the skin of a human or animal wearer. For example, in the case of a protective cover/pouch having multilayers, a portion of a layer thereof intended to be in contact with a patient's skin can include a skin-safe adhesive.

Thus, it is seen that the objects of the invention are efficiently obtained, although modifications and changes to the invention may be readily imagined by those having ordinary skill in the art, and these changes and modifications are intended to be within the scope of the claims.

| | | | | | |
|---|---|---|---|---|---|
| 5 | Adhesive Tape | 10 | Patient/Patient Arm | 15 | Catheter Assembly |
| 20 | Catheter Port | 30 | Catheter Hub | 40 | Catheter Insertion Point |
| 50 | Clear Adhesive | 60 | Tube | 100 | Protective Cover/Pouch |
| | Bandage | | | | |
| 102 | Cover/Guard Portion | 104 | First End | 106 | Second End |
| 108 | Pouch Assembly | 110 | Sleeve | 112 | Window/Viewing |
| | Portion | | | | Portion |
| 120 | Window/Aperture | 130 | Pocket | 140 | Window/Aperture |
| | Cover | | | | |
| 150 | Fastener | 160 | Fastener | 170 | Hinge |
| 175 | Wall/Side Wall | 180 | Slit/Aperture | 190 | Elastic Band |
| 195 | Fastener | 200 | Fastener | 210 | Anti-slip Portion |
| | | | | | (Rubber/Silicone) |
| 220 | External | 230 | Internal | 300 | Protective Cover/Pouch |
| | Surface/Layer//Fabric | | Surface/Layer/Fabric | | |
| 310 | Strap/Band | 320 | Buckle | 330 | Cutaway/Hidden View |
| 400 | Protective Cover/Pouch | 410 | Pull Tab | 420 | Medical Indicia/Design |
| 430 | Overlapping Flap | 440 | Breathable | 500 | Protective Cover/Pouch |
| | | | Fabric/Netting | | |
| 510 | Pocket | 520 | Zipper Assembly | 600 | Protective Cover/Pouch |
| 610 | Button/Snap | 700 | Protective Cover/Pouch | 710 | Window |
| 800 | Protective Cover/Pouch | 900 | Protective Cover/Pouch | 1000 | Protective Cover/Pouch |

What is claimed is:

1. A protective cover/pouch assembly detachably securable to a wearer, the protective cover/pouch having a first end and a second end, comprising:
a cover/guard portion having an inner surface and an outer surface, the inner surface configured to be disposed proximate the wearer and the outer surface configured to be disposed distal the wearer;
a pouch assembly portion disposed between the first end and the second end, the pouch assembly including a cavity disposed at one or more of:
between the inner surface and the outer surface of the cover/guard portion,
on the inner surface of the cover/guard portion, or
on the outer surface of the cover/guard portion,
wherein the cavity is accessible via an exterior opening in an outer oriented surface of the pouch assembly portion, the exterior opening openable and closable via one or more of an elastically deformable member, an overlapping flap assembly, a zipper, a hook and loop-type fastener assembly, one or more snaps or buttons, a cinching member, or a re-sealable sliding channel sealing assembly;
wherein, the cavity is accessible via at least one interior slit/aperture disposed in an inner oriented surface of the pouch assembly portion; and,
wherein, one or more of the cover/guard portion and the pouch assembly portion comprises a stretchable and elastically deformable material that conforms with a portion of the wearer's body.

2. The protective cover/pouch assembly of claim 1, wherein the inner surface of the cover/guard portion comprises the stretchable and elastically deformable material, the cover/guard portion formed in the manner of a sleeve configured to receive the portion of the wearer's body.

3. The protective cover/pouch assembly of claim 1, comprising at least one strap member.

4. The protective cover/pouch assembly of claim 1, wherein at least one of the cover/guard portion and/or the pouch assembly portion comprises one or more of a breathable fabric, a moisture wicking fabric, an anti-microbial fabric, and a cushioning material.

5. The protective cover/pouch assembly of claim 4, wherein at least one of the cover/guard portion and/or the pouch assembly portion comprises at least one of a mesh or a plurality of vent holes.

6. The protective cover/pouch assembly of claim 5, wherein at least one of the cover/guard portion and/or the pouch assembly portion comprise a foam.

7. The protective cover/pouch assembly of claim 1, wherein the cavity of the pocket assembly portion is formed from at least one side wall that extends from the outer surface of the cover/guard portion.

8. The protective cover/pouch assembly of claim 1, wherein the at least one side wall comprises at least one of a breathable fabric, a moisture wicking fabric, an anti-microbial fabric, and a cushioned fabric.

9. The protective cover/pouch assembly of claim 8, wherein the at least one side wall comprises at least one of a mesh or a plurality of vent holes.

10. The protective cover/pouch assembly of claim 1, further comprising a viewing window/region disposed between the first end and the second end,
wherein, the viewing window/region is disposed toward the first end and the pouch assembly region is disposed toward the second end;
wherein, the viewing window/region comprises an aperture formed in the cover/guard portion; and
wherein, the aperture is accessible via an openable and closable aperture cover disposed over the aperture.

11. The protective cover/pouch assembly of claim 10, wherein:
the aperture and the aperture cover are configured to be disposed over a catheter hub portion of a mounted catheter assembly, and the pouch assembly portion is configured to be disposed over a catheter port portion of the mounted catheter assembly; and,
the catheter port portion is configured to be passed through the at least one slit/aperture disposed in the inner oriented surface of the pouch assembly portion and received within the cavity.

12. The protective cover/pouch assembly of claim 10, wherein the aperture cover includes a flap portion and a hinge portion, the hinge portion secured to the cover/guard portion.

13. The protective cover/pouch assembly of claim 12, wherein the hinge portion is oriented transverse a longitudinal axis of the protective cover/pouch assembly, or oriented parallel the longitudinal axis.

14. The protective cover/pouch assembly of claim 10, wherein the aperture cover includes at least one male/female fastener assembly, and a portion of the cover/guard portion bordering the aperture includes a corresponding at least one female/male fastener assembly that complementarily fastens to the male/female fastener assembly of the aperture cover.

15. The protective cover/pouch assembly of claim 14, wherein the at least one male/female fastener and corresponding at least one female/male fastener assembly comprise at least one or more of hook and loop, a zipper, a snap, or a button.

16. The protective cover/pouch assembly of claim 14, wherein the aperture cover includes at least one pull tab allowing the aperture cover to be gripped.

17. The protective cover/pouch assembly of claim 10, wherein the aperture cover includes indicia indicating the potential that a medical device or a medical component is disposed below the aperture cover.

18. The protective cover/pouch assembly of claim 10, wherein the at least one of the cover/guard, the pocket, or the aperture cover comprises one or more of a skin-tone hue, a hue corresponding to one or more colors, a pattern, a design, or artwork.

19. The protective cover/pouch assembly of claim 10, further comprising an elastic band portion disposed proximate the first end, the elastic band conforming to a shape of a portion of the wearer's body.

20. The protective cover/pouch assembly of claim 10, further comprising a non-slip member disposed on the inner surface of the cover/guard.
